(19) United States
(12) Patent Application Publication
(10) Pub. No.: US 2010/0234733 A1
(43) Pub. Date: Sep. 16, 2010

(54) Sterile Ultrasound Probe Cover and Method of Releasing Coupling Agent from a Sealed Compartment

(76) Inventor: Paul Wahlheim, Phoenix, AZ (US)

Correspondence Address: Robert D. Atkins, 605 W. Knox Road, Suite 104, Tempe, AZ, 85284, US

(21) Appl. No.: 12/404,156

(22) Filed: Mar. 13, 2009

(51) Int. Cl. A61B 8/14

(52) U.S. Cl. 600/459

(57) ABSTRACT

A sterile cover tube for ultrasound probe and electrical cable has a telescoping tube having a closed end and open end. The telescoping tube is extendable to cover the ultrasound probe and electrical cable. A sealed pouch containing coupling agent is disposed adjacent to the closed end of the telescoping tube. The sealed pouch has a breakable seam or membrane. The sealed pouch can be placed over the probe head prior to placing the cover tube over the probe and electrical cable. The sealed pouch is ruptured by pressure to release the coupling agent over the ultrasound probe after the telescoping tube has been extended over the ultrasound probe and electrical cable. The coupling agent covers the probe head with a thickness of 0.2-6.0 millimeters after the sealed pouch is ruptured to provide an airless medium for an accurate image of the body region of interest.

### FIELD OF THE INVENTION

The present invention relates in general to medical devices and, more particularly, to a sterile cover tube and sealed compartment containing a coupling agent for maintaining a sterile environment for the transducer probe head and electrical cable of a medical ultrasound unit. The sterile cover tube and sealed compartment provides optimal orientation of the coupling agent and ultrasound probe head.

### BACKGROUND OF THE INVENTION

An ultrasound instrument uses a cyclic mechanical sound or pressure wave, with a frequency typically greater than 20 kHz, and has extensive applications in medical diagnosis and treatment. A health care provider places the ultrasound instrument over an area of interest on the human body. The instrument generates sound or pressure waves which penetrate the soft tissue of the body. The pressure waves incident to an internal structure of interest in the body are reflected back. The instrument receives the reflected waves and converts the data into electrical signals, which are routed to a computer analysis system. The ultrasound measurements reveal detail of the inner structures, such as size, depth, composition, and orientation in real-time images. Ultrasound allows the health care provider to image muscles, tendons, blood vessels, and internal organs, such as the heart, respiratory system, and digestive system. Ultrasound has applications in cardiology, OB/GYN, radiology, urology, internal medicine, emergency medicine, breast imaging, and anesthesiology. For example, ultrasound can be used during the third trimester to monitor and analyze the condition of the fetus, e.g., gestational age, fetal viability, physical abnormalities, fetal growth, fetal movement and heartbeat, and sex of the fetus, as part of routine and diagnostic prenatal care. Another example, is sterile central venous access in emergency medicine.

Ultrasound testing is typically done in a sterile manner. U.S. Pat. No. 5,259,383 describes an ultrasound instrument which includes a non-sterile housing containing a transducer probe and electrical cable connected to a computer analysis system. The transducer probe generates the mechanical sound or pressure waves which are emitted into the area of interest in the body. The sound or pressure waves incident to an internal structure of interest are reflected back. The reflected pressure waves are received by the probe and converted to electrical signals. The electrical cable routes the electrical signals to the computer analysis system, which displays a visual image of the body area of interest.

To maintain a sterile environment, a sterile cover tube is placed over the transducer probe. The cover tube is about 2.5 meters long and completely covers the probe and a portion of the electrical cable that may come in contact with the sterile field, e.g., patient covering. The sterile cover tube may extend along the entire electrical cable up to the connector of the computer analysis system. The cover tube has a closed end and an open end. A coupling agent is provided in gel form in a separate packet, tube, or other container. Prior to placing the cover tube over the probe, the operator removes the coupling agent from the separate container and applies the agent liberally over the probe head or the inside surface of the closed end of the cover tube. After applying the coupling agent, the cover tube is pulled over the transducer probe and electrical cable, as carefully as possible, while attempting to not disturb the thickness or coverage area of the coupling agent between the transducer probe head and closed end of the cover tube. In an ideal situation, the coupling agent maintains a continuous, airless transmission medium between the cover tube and probe head.

However, if the coupling agent is disturbed, thinned, or removed from part or all of the transducer probe head while placing the cover tube over the instrument, then the pressure wave transmission path is degraded resulting in poor image quality and resolution. The ultrasound test may become inaccurate and possibly lead to an erroneous diagnosis or unnecessary procedural challenges. The task of placing the cover tube over the probe head without disturbing or losing coverage of coupling agent is difficult and often requires multiple personnel to handle the procedure. The procedure can be messy as the coupling agent is invariably transferred to or tracked along the probe housing, electrical cable, and inside portion of the cover tube. Clean-up after the ultrasound procedure is time consuming.

### SUMMARY OF THE INVENTION

A need exists for an efficient and effective manner of applying coupling agent between the transducer probe head and cover tube. Accordingly, in one embodiment, the present invention is a sterile cover tube for an ultrasound probe and electrical cable comprising a telescoping tube having a closed end and an open end. The telescoping tube is extendable to cover the ultrasound probe and electrical cable. A sealed pouch containing coupling agent is disposed adjacent to the closed end of the telescoping tube. The sealed pouch is ruptured to release the coupling agent over the ultrasound probe after the telescoping tube has been extended over the ultrasound probe and electrical cable.

In another embodiment, the present invention is a medical instrument cover for enclosing an ultrasound probe and electrical cable comprising a cover tube having a closed end and an open end. The cover tube is extendable to cover the ultrasound probe and electrical cable. A sealed compartment containing coupling agent is disposed adjacent to the closed end of the cover tube. The sealed pouch is ruptured to release the coupling agent over the ultrasound probe after the cover tube has been extended over the ultrasound probe and electrical cable.

In another embodiment, the present invention is a medical instrument cover for enclosing an ultrasound probe and electrical cable comprising a tube having a closed end and an open end. A sealed compartment containing coupling agent is disposed adjacent to the closed end of the cover tube.

In another embodiment, the present invention is a method of covering an ultrasound probe and electrical cable comprising the steps of providing a telescoping tube having a closed end and an open end, disposing a sealed compartment containing coupling agent adjacent to the closed end of the telescoping tube, extending the telescoping tube to cover the ultrasound probe and electrical cable, and rupturing the sealed pouch to release the coupling agent over the ultrasound probe after the telescoping tube has been extended over the ultrasound probe and electrical cable.

### BRIEF DESCRIPTION OF THE DRAWINGS

FIG. 1 illustrates ultrasound equipment to give a patient an ultrasound diagnosis procedure;

FIGS. 2a-2c illustrate various embodiments of the transducer probe head and electrical cable;

FIGS. 3a-3b illustrate embodiments of a sterile telescoping cover tube with sealed pouch(s) containing coupling agent disposed adjacent to the closed-end of the tube;

FIGS. 4a-4c illustrate placing the telescoping cover tube with sealed pouch containing coupling agent over transducer head and electrical cable;

FIG. 5 illustrates a breakable seam or membrane in the sealed pouch for releasing coupling agent under pressure;

FIG. 6 illustrates a sealed pouch containing coupling agent disposed over the probe head prior to placing the cover tube over the probe and electrical cable; and

FIG. 7 illustrates a solid form of coupling agent disposed on the closed-end of the cover tube.

### DETAILED DESCRIPTION OF THE DRAWINGS

The present invention is described in one or more embodiments in the following description with reference to the Figures, in which like numerals represent the same or similar elements. While the invention is described in terms of the best mode for achieving the invention's objectives, it will be appreciated by those skilled in the art that it is intended to cover alternatives, modifications, and equivalents as may be included within the spirit and scope of the invention as defined by the appended claims and their equivalents as supported by the following disclosure and drawings.

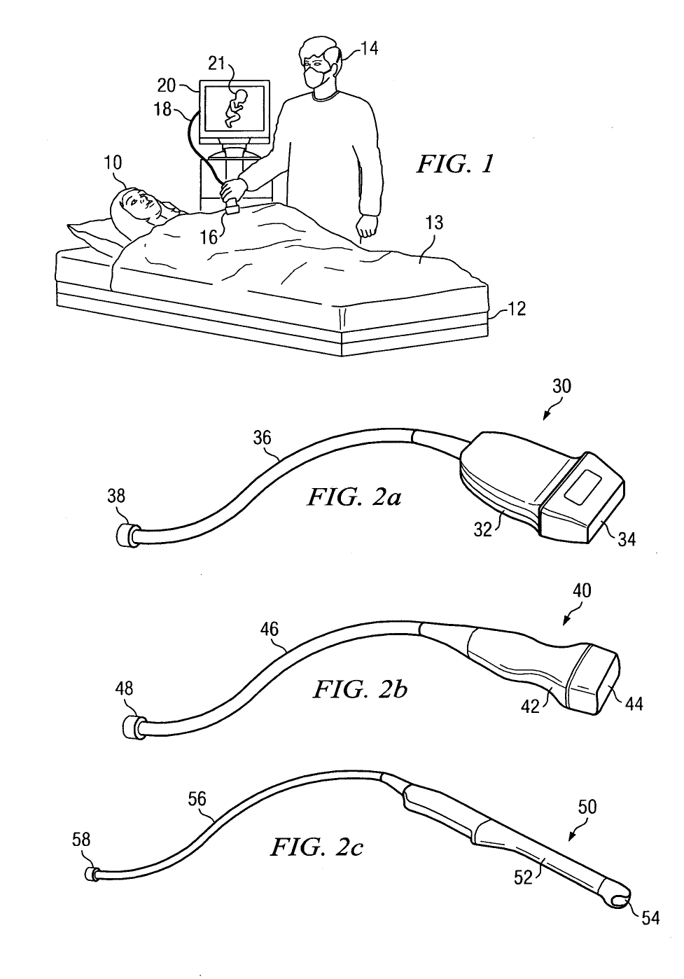

Turning to FIG. 1, a patient 10 is shown undergoing an ultrasound testing procedure. Patient 10 rests on bed or examination table 12 with sterile covering 13 to form a sterile field. Health care provider or operator 14 stands or sits adjacent to patient 10. Operator 14 holds ultrasound transducer probe 16 in his or her hand. Probe 16 is connected by electrical cable 18 to computer analysis system 20. Operator 14 places probe 16 over the soft tissue over the area of interest of patient 10. For example, operator 14 places probe 16 over the abdomen of patient 10. Probe 16 emits a cyclic mechanical sound or pressure wave, with a frequency typically greater than 20 kHz. The pressure wave travels through the body tissue to the internal region or structure of interest. The pressure wave incident to the region of interest is reflected back to the probe. The reflected pressure wave is converted to an electrical signal by the transducer probe which provides a representation of the inner structure of interest. The electrical signal is routed to computer analysis system 20, which performs signal processing on the electrical signals and displays a visual representation or image 21 of the internal structure of interest.

The ultrasound testing procedure allows the health care provider to image muscles, tendons, blood vessels, and internal organs, such as the heart, respiratory system, and digestive system. The images can help determine physical attributes of the region of interest including size, structure, and abnormalities in real-time tomographic images. Ultrasound has applications in many medical fields such as cardiology, OB/GYN, internal medicine, radiology, urology, emergency medicine, breast imaging, and anesthesiology, including procedures for intracorporeal, intraoperative ultrasonography, ultrasound guided biopsy techniques, pericardialcentesis, central venous access, thoracostomy, and ultrasound guided central line placement. For example, ultrasound can be used during the third trimester to monitor and analyze the condition of the fetus, e.g., gestational age, fetal viability, physical abnormalities, fetal growth, fetal movement and heartbeat, and sex of the fetus, as part of routine and diagnostic prenatal care.

FIG. 2a illustrates an exemplary transducer probe 30 including housing 32 containing pressure wave transceiver 34 and electronic circuits for pressure wave generation, sensors, transducer, filtering, and amplification. An electrical cable 36 and connector 38 connects transducer probe 30 to computer analysis system 20.

FIG. 2b shows another transducer probe 40 including housing 42 containing pressure wave transceiver 44 and electronic circuits for pressure wave generation, sensors, transducer, filtering, and amplification. An electrical cable 46 and connector 48 connects transducer probe 40 to computer analysis system 20.

FIG. 2c shows another transducer probe 50 including housing 52 containing pressure wave transceiver 54 and electronic circuits for pressure wave generation, sensors, transducer, filtering, and amplification. An electrical cable 56 and connector 58 connects transducer probe 50 to computer analysis system 20.

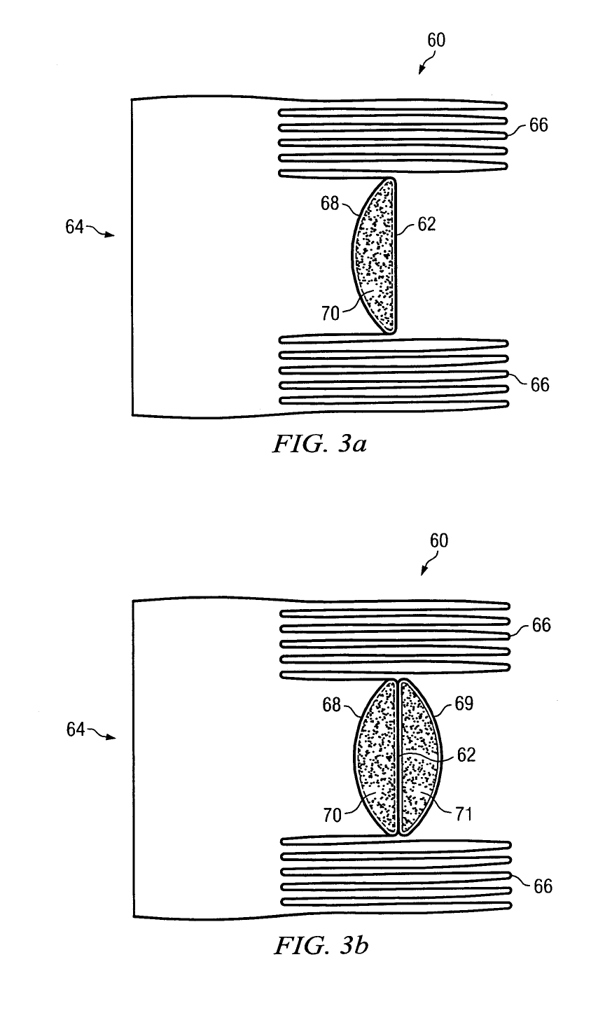

In an effort to limit central line related infections, generally accepted recommendations call for a sterile ultrasound guided central line placement. Accordingly, ultrasound imaging is typically performed in a sterile manner. Since the transducer probe and electrical cable shown in FIGS. 2a-2c are non-sterile, a sterile cover must be placed over the instrument. FIG. 3a shows a sterile cover tube 60 including closed-end 62 and open-end 64. In one embodiment, cover tube 60 is a radiosterilizable plastic foil tube of high-density and low-density polyethylene and copolymers of ethyl/butyl acrylate or ethylene/methyl acrylate (EMA). Cover tube 60 has a diameter of 14 centimeters (cm) and material thickness of 15 micrometers (μm) to 50 μm. Cover tube 60 is made of sufficient length, typically 2.5 meters, to completely cover the transducer probe and a portion of electrical cable that may come in contact with the sterile field, e.g., patient covering. In some cases, cover tube 60 extends up to the connector of computer analysis system 20. In its initial form, sidewalls 66 of cover tube 60 are telescopically folded over each other in short sections to reduce the length of the tube to a manageable size. Each telescopic fold has length of 25 cm and are mutually superimposed radially to the longitudinal axis of cover tube 60.

The inside surface of closed-end 62 includes pouch or compartment 68 containing coupling agent or medium 70. Pouch 68 is attached or otherwise disposed adjacent to the inside surface of closed-end 62 of cover tube 60 with an adhesive. The adhesive seam of pouch 68 is located on the lateral sidewalls of cover tube 60, i.e., just outside closed-end 62, to avoid impairing the transmission path directly in-line with the pressure wave transceiver. Coupling agent 70 is a hypo-allergenic, water-soluble, greaseless, high transmission conductivity, non-staining gel or physiological saline solution. The primary purpose of the coupling agent or medium is to facilitate transmission of the ultrasound energy from the probe head to tissue and back again. The greater the difference in impedance at a boundary, the smaller the amount of energy that will be transferred. Accordingly, coupling agent 70 and the lining of cover tube 60 are designed to match the impedance of human tissue.

The outside surface of closed-end 62 may also include a pouch or compartment 69 containing coupling agent or medium 71, as shown in FIG. 3b. Pouch 69 is attached or otherwise disposed adjacent to the outside surface of closed-end 62 of cover tube 60 with an adhesive. The adhesive seam of pouch 69 is located on the lateral sidewalls of cover tube 60, i.e., just outside closed-end 62, to avoid impairing the transmission path directly in line with the pressure wave transceiver. Coupling agent 71 is a hypo-allergenic, water-soluble, greaseless, high transmission conductivity, non-staining gel or physiological saline solution. The primary purpose of the coupling agent or medium is to facilitate transmission of the ultrasound energy from the probe head to tissue and back again.

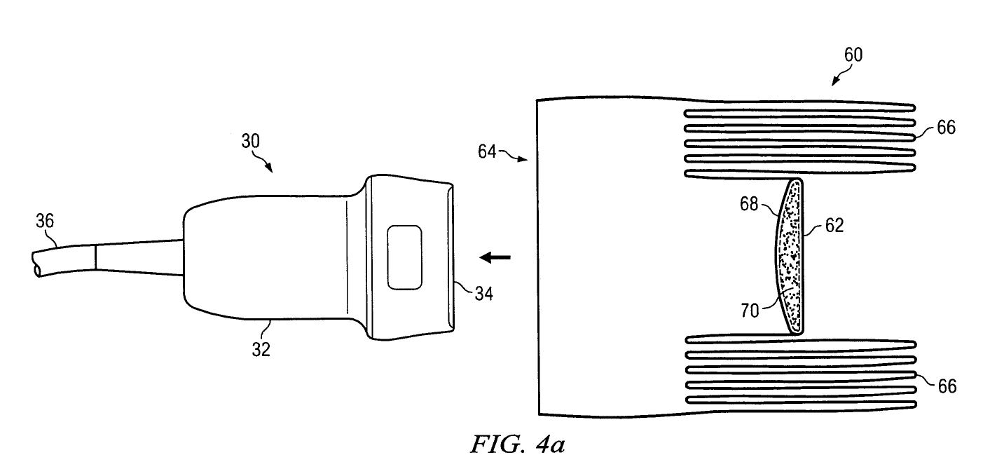

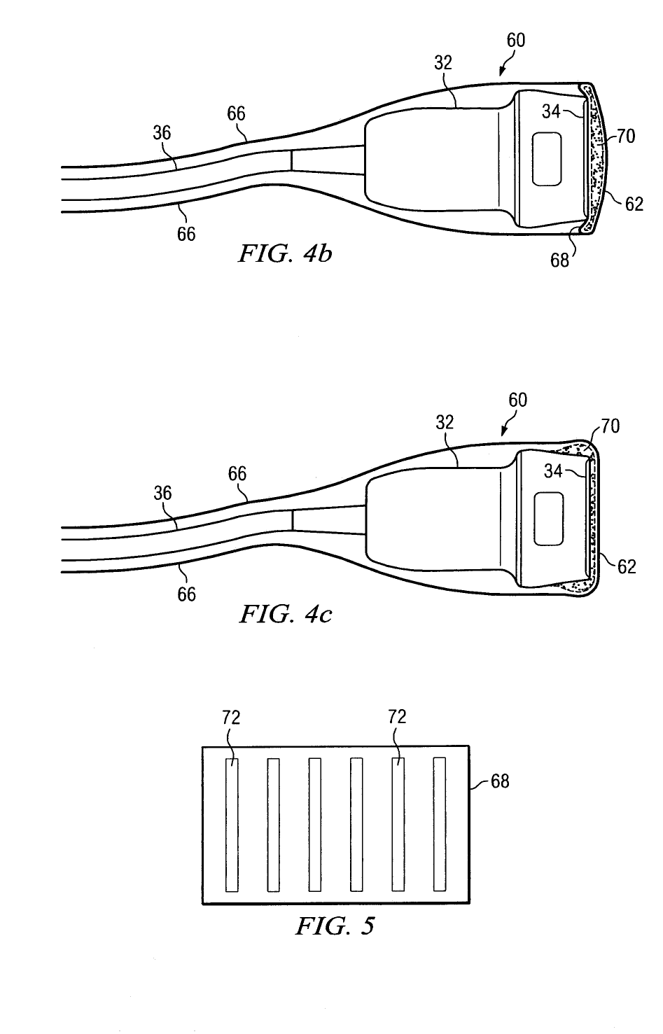

In FIG. 4a, transducer probe 30 is placed adjacent to cover tube 60. Using the embodiment from FIG. 2a and leading with pressure wave transceiver 34, probe 30 is inserted through open-end 64 such that the surface of pressure wave transceiver 34 contacts pouch 68. Coupling agent 70 remains within sealed pouch 68 at this time.

With the pressure wave transceiver contacting sealed pouch 68, sidewalls 66 of cover tube 60 are elongated in a telescoping manner away from transducer probe 30 to cover electrical cable 36, as shown in FIG. 4b. The sterile cover tube 60 extends over non-sterile housing 32 and electrical cable 36 beyond potential contact with the sterile field, possibly up to computer analysis system 20 to maintain a sterile field. Since coupling agent 70 remains in sealed pouch 68, the coupling agent is not disturbed nor is it inadvertently transferred to or tracked along sidewalls 66 as cover tube 60 is unfolded.

In FIG. 4c, with cover tube 60 securely in place over transducer probe 30 and electrical cable 36, pouch 68 is ruptured to release coupling agent 70 over pressure wave transceiver 34. Pouch 68 contains a breakable seam or membrane. In one embodiment, a plurality of parallel breakable seams 72 runs along a surface of closed-end 62 adjacent to pressure wave transceiver 34, as shown in FIG. 5. Breakable seams 72 are relatively thin material which is easily broken with application of pressure, e.g., by pressing against the pouch with the fingers or palm. Sealed pouch 68 is thus manually ruptured along breakable seam 72 when pressure is applied to release coupling agent 70. The tensile strength of breakable seam 72 of pouch 68 is less than the lining of cover tube 60. In one embodiment, the lining of cover tube 60 has twice the tensile strength of the breakable seam 72. Accordingly, cover tube 60 remains intact when pouch 68 is ruptured. The position of coupling agent and discrepancy in tensile strength of pouch 68 and outer tube cover provides for optimal positioning of coupling agent 70 after manual rupture of the pouch. Coupling agent 70 is dispersed over pressure wave transceiver 34 to the proper thickness and coverage. In the case of FIG. 3b, pouch 69 is also ruptured by the pressure and coupling agent 71 is dispersed over the patient within the sterile field. In one embodiment, coupling agent 70 ranges from 0.2-6.0 millimeters (mm) in thickness to negate any air pockets and provide an airless medium between pressure wave transceiver 34 and closed-end 62 of cover tube 60 for optimal visualization of the regional anatomy of interest, while the sterile cover tube decreases the possibility of infection. Coupling agents 70 and 71 are not disturbed and remain within the narrow confines of pressure wave transceiver 34 because cover tube 60 has already been elongated to obtain the sterile field prior to dispersement of the coupling agent. The ultrasound test is conducted with the proper concentration and coverage of coupling agents 70 and 71 over pressure wave transceiver 34 and patient 10. The visual image displayed on computer analysis system 20 is an accurate representation of the body region of interest.

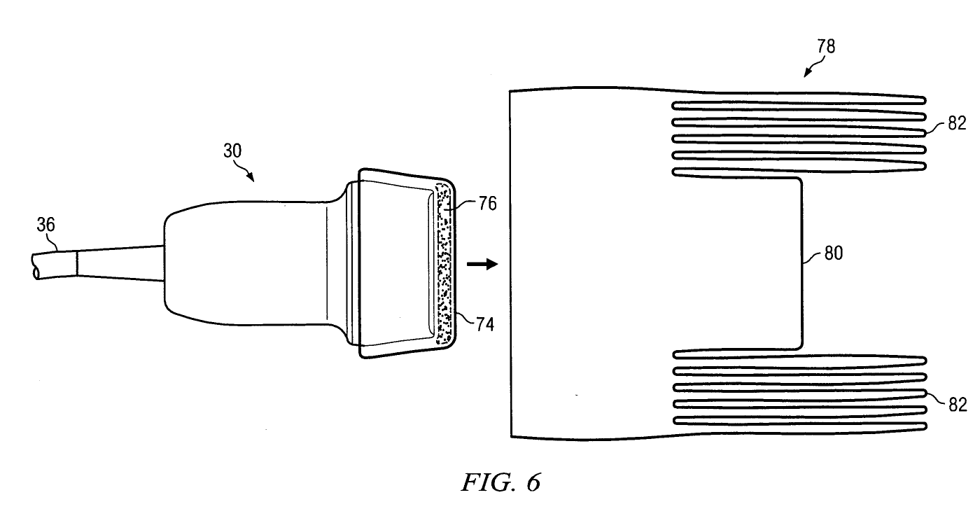

Another embodiment of the medical instrument cover is shown in FIG. 6. A separate pouch or compartment 74 is placed over pressure wave transceiver 34 of transducer probe 30 prior to placing the cover tube over the probe. Pouch 74 contains coupling agent or medium 76. Cover tube 78 is then placed over transducer probe 30, similar to FIG. 4a-4c. Probe 30 is inserted through the open-end of cover tube 78 such that the surface of pressure wave transceiver 34, with sealed pouch 74, contacts closed-end 80. Coupling agent 76 remains within sealed pouch 74 at this time.

With sealed pouch 74 contacting closed-end 80, sidewalls 82 of cover tube 78 are elongated in a telescoping manner away from transducer probe 30 to cover electrical cable 36. The sterile cover tube 78 extends over non-sterile housing 32 and electrical cable 36 beyond potential contact with the sterile field, possibly up to computer analysis system 20 to maintain a sterile field. Since coupling agent 76 remains in sealed pouch 74, the coupling agent is not disturbed nor is it inadvertently transferred to or tracked along sidewalls 82 as cover tube 78 is unfolded.

With cover tube 78 securely in place over transducer probe 30 and electrical cable 36, pouch 74 is ruptured to release coupling agent 76 over pressure wave transceiver 34. Pouch 78 contains a breakable seam or membrane, similar to FIG. 5, on one or both sides of the pouch. The sealed pouch is manually ruptured along the breakable seam when pressure is applied, e.g., by pressing the pouch with the fingers or palm, to release coupling agent 76. The tensile strength of the breakable seam or membrane is less than the lining of cover tube 78. Accordingly, cover tube 78 remains intact when sealed pouch 74 is ruptured. Coupling agent 76 is dispersed over pressure wave transceiver 34 to the proper thickness and coverage. In one embodiment, coupling agent 70 ranges from 0.2-6.0 mm in thickness to negate any air pockets and provide an airless medium between pressure wave transceiver 34 and closed-end 80 of cover tube 78 for optimal visualization of regional anatomy, while the sterile cover tube decreases the possibility of infection. Coupling agent 76 is not disturbed and remains within the narrow confines of pressure wave transceiver 34 because cover tube 78 has already been elongated to obtain the sterile field prior to dispersement of the coupling agent. The ultrasound test is conducted with the proper concentration and coverage of coupling agent 76 over pressure wave transceiver 34. The visual image displayed on computer analysis system 20 is an accurate representation of the body region of interest.

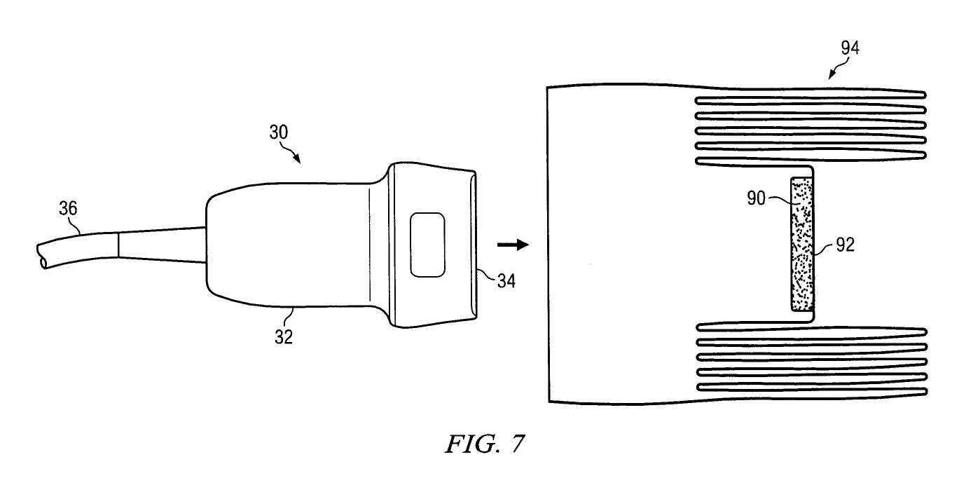

In another embodiment, a coupling agent 90 is placed directly on closed-end 92 of cover tube 94, as shown in FIG. 7. Coupling agent 90 is in solid or semi-solid form when placed on closed-end 92. Alternatively, the solid or semi-solid coupling agent is placed on the pressure wave transceiver 34. While coupling agent 90 is in a solid or semi-solid form, cover tube 94 is placed over probe 30 and electrical cable 36, similar to FIGS. 4a-4c. Coupling agent 90 is then heated to a gel state. The gel form of coupling agent 90 disperses over pressure wave transceiver 34 to the proper thickness and coverage. In one embodiment, coupling agent 90 ranges from 0.2-6.0 mm in thickness to negate any air pockets and provide an airless medium between pressure wave transceiver 34 and closed-end 92 of cover tube 94 for optimal visualization of regional anatomy, while the sterile cover tube decreases the possibility of infection. Coupling agent 90 is not disturbed and remains within the narrow confines of pressure wave transceiver 34 because cover tube 78 has already been elongated to obtain the sterile field prior to heating the solid form of coupling agent 90 into a gel state. The ultrasound test is conducted with the proper concentration and coverage of coupling agent 90 over the pressure wave transceiver. The visual image displayed on computer analysis system 20 is an accurate representation of the body region of interest.

While one or more embodiments of the present invention have been illustrated in detail, the skilled artisan will appreciate that modifications and adaptations to those embodiments may be made without departing from the scope of the present invention as set forth in the following claims.

What is claimed:

1. A sterile cover tube for an ultrasound probe and electrical cable, comprising:
a telescoping tube having a closed end and an open end, the telescoping tube being extendable to cover the ultrasound probe and electrical cable; and
a sealed pouch containing coupling agent disposed adjacent to the closed end of the telescoping tube, wherein the sealed pouch is ruptured to release the coupling agent over the ultrasound probe after the telescoping tube has been extended over the ultrasound probe and electrical cable.

2. The sterile cover tube of claim 1, wherein the sealed pouch includes a breakable seam or membrane.

3. The sterile cover tube of claim 1, wherein the sealed pouch is ruptured by application of pressure.

4. The sterile cover tube of claim 1, wherein the coupling agent covers the ultrasound probe to a thickness of 0.2-6.0 millimeters after the sealed pouch is ruptured.

5. The sterile cover tube of claim 1, wherein the coupling agent includes gel or saline solution.

6. A medical instrument cover for enclosing an ultrasound probe and electrical cable, comprising:
a cover tube having a closed end and an open end, the cover tube being extendable to cover the ultrasound probe and electrical cable; and
a sealed compartment containing coupling agent disposed adjacent to the closed end of the cover tube, wherein the sealed compartment is ruptured to release the coupling agent over the ultrasound probe after the cover tube has been extended over the ultrasound probe and electrical cable.

7. The medical instrument cover of claim 6, wherein the sealed compartment includes a breakable seam or membrane.

8. The medical instrument cover of claim 6, wherein the sealed compartment is ruptured by application of pressure.

9. The medical instrument cover of claim 6, wherein the coupling agent covers the ultrasound probe to a thickness of 0.2-6.0 millimeters after the sealed compartment is ruptured.

10. The medical instrument cover of claim 6, wherein the coupling agent includes gel or saline solution.

11. The medical instrument cover of claim 6, wherein the cover tube is telescoping to extend over the ultrasound probe and electrical cable.

12. A medical instrument cover for enclosing an ultrasound probe and electrical cable, comprising:
a tube having a closed end and an open end; and
a first sealed compartment containing coupling agent disposed adjacent to a first side of the closed end of the tube.

13. The medical instrument cover of claim 12, wherein the tube is extendable to cover the ultrasound probe and electrical cable and the first sealed compartment is ruptured to release the coupling agent over the ultrasound probe after the tube has been extended over the ultrasound probe and electrical cable.

14. The medical instrument cover of claim 12, wherein the first sealed compartment includes a breakable seam or membrane.

15. The medical instrument cover of claim 12, wherein the first sealed compartment is ruptured by application of pressure.

16. The medical instrument cover of claim 12, wherein the coupling agent covers the ultrasound probe to a thickness of 0.2-6.0 millimeters after the first sealed compartment is ruptured.

17. The medical instrument cover of claim 12, wherein the coupling agent includes gel or saline solution.

18. The medical instrument cover of claim 12, wherein the first sealed compartment is placed over a head of the probe head prior to placing the cover tube over the probe and electrical cable.

19. The medical instrument cover of claim 12, wherein the cover tube is telescoping to extend over the ultrasound probe and electrical cable.

20. The medical instrument cover of claim 12, further including a second sealed compartment containing coupling agent disposed adjacent to a second side of the closed end of the tube opposite the first side of the closed end of the tube.

21. A method of providing sterile covering for an ultrasound probe and electrical cable, comprising:
providing a telescoping tube having a closed end and an open end;
disposing a sealed compartment containing coupling agent adjacent to the closed end of the telescoping tube;
extending the telescoping tube to cover the ultrasound probe and electrical cable; and
rupturing the sealed compartment to release the coupling agent over the ultrasound probe after the telescoping tube has been extended over the ultrasound probe and electrical cable.

22. The method of claim 21, further including forming a breakable seam or membrane in the sealed compartment.

23. The method of claim 21, further including applying pressure to rupture the sealed compartment.

24. The method of claim 21, wherein the coupling agent includes gel or saline solution.

25. The method of claim 21, wherein the cover tube is telescoping to extend over the ultrasound probe and electrical cable.
